(19) United States
(12) Patent Application Publication
(10) Pub. No.: US 2005/0215875 A1
(43) Pub. Date: Sep. 29, 2005

(54) Method and device for locating position of veins on body

(76) Inventor: Sroy Khou, La Punete, CA (US)

Correspondence Address: RAYMOND Y. CHAN, 108 N. YNEZ AVE., SUITE 128, MONTEREY PARK, CA, 91754, US

(21) Appl. No.: 10/804,917

(22) Filed: Mar. 19, 2004

(51) Int. Cl. A61B 5/05; A61B 6/00

(52) U.S. Cl. 600/407; 600/476; 600/473

(57) ABSTRACT

A vein position locating device includes a vein probing head which includes two light emitters spacedly and opposedly apart from each other to define a treatment channel therebetween, wherein the vein probing head is adapted for contacting on a skin of a user so that each of the light emitters is adapted for emitting a light beam to penetrate through the skin of the user so as to visualize a vein thereunder. The vein probing head is arranged for moving on the skin of the user until the vein is aligned in the treatment channel between the two light emitters for vein treatment.

### BACKGROUND OF THE PRESENT INVENTION

1. Field of Invention

The present invention relates to a medical device, and more particularly to a method and device for locating position of veins on a body so as to facilitate faster and more accurate positioning of veins for more effective medical treatment, such as more accurate injections.

2. Description of Related Arts

Injections have become one of the most common forms of medical treatments which provide generally rapid and effective remedies to a wide variety of diseases or illnesses. The usual practice is that when a medical doctor has diagnosed that a particular dose of medicine need being injected to a particular patient, a nurse will carry out the injection as prescribed by the doctor.

There are generally four steps in carrying out the required injection. First, preparing a predetermined amount of medicine as prescribed by the doctor in the syringe; second, locating the vein which is to be injected with the medicine; third, injecting the medicine to the vein through breaking the skin right above the vein; and fourth, removing the syringe from the body.

Thus one can see that a typical injection procedure, though frequently performed, involves a high level of specialized skill which can only be acquired by months or years of supervised training. During the training period, the patients to which the training is targeted would risk the possibility of poor injection technique and inadequate clinical experience possessed by the trainee.

Even for a qualified nurse who is entitled to perform injection, due to the very nature by which the injection procedure owes, i.e. manual operation and subjective judgment as to the, say, position of the vein, so the quality of the injection varies from one nurse to the another. As a result, it is extremely difficult, and indeed virtually impossible, to maintain a consistent standard of clinical injection practice within one department, such as the casualty department, of even a small hospital. This is the reason why one might frequently hear of handfuls of medical errors involving injections from daily newspapers.

Notwithstanding such deep-seated problem, there seems to be no effective remedy for addressing it. In fact, there exist some kinds of medical accessories, such as a tightening band, which is adapted to tighten a predetermined part of a patient's body in order to narrow the veins therearound. After that, since the some portions of the veins are tightened, the untightened part of the veins in the vicinity of the tightened part will be expanded and, as a result, allowing the nurse to observe the vein clearer for injection. The effectiveness and performance of these kinds of accessories must be questioned. Moreover, for some accessories, especially those needing to wear on the patient's arm, they are very inconvenient to be used in practice.

### SUMMARY OF THE PRESENT INVENTION

A main object of the present invention is to provide a vein position locating device for rapidly and accurately locating a vein of a user such that an effective medical treatment, such as a more accurate injection, can be accomplished.

Another object of the present invention is to provide a vein position locating device comprising a vein probing head which is adapted to move on a user's skin for accurately and rapidly locating a vein thereof by virtue of two light emitters. In other words, no destruction to the skin is needed for locating the user's vein.

Another object of the present invention is to provide a vein position locating device which does not involve any complicated or expensive mechanical or electrical components so as to minimize the manufacturing cost and the ultimate selling price of the present invention. In other words, the present invention substantially overcomes a traditional scenario that medical equipments tend to be expensive.

Another object of the present invention is to provide a method of locating a vein under a user's skin so as to facilitate faster and more accurate positioning of veins for more effective medical treatment, such as more accurate injections.

Another object of the present invention is to provide a method of locating a vein under a user's skin by the vein position locating device which does not incur any damages to the user's skin.

Accordingly, in order to accomplish the above objects, the present invention provides a vein position locating device, comprising:
a vein probing head which comprises two light emitters spacedly and opposedly apart from each other to define a treatment channel therebetween, wherein the vein probing head is adapted for contacting on a skin of a user so that each of the light emitters is adapted for emitting a light beam to penetrate through the skin of the user so as to visualize a vein thereunder, wherein the vein probing head is arranged for moving on the skin of the user until the vein is aligned in the treatment channel between the two light emitters for vein treatment.

Moreover, the present invention further provides a method of locating a vein under a user's skin by a vein position locating device which comprises a vein probing head comprising two light emitters which define a treatment channel, wherein the vein locating method comprises the steps of:
(a) locating the vein probing head on the user's skin wherein the two light emitters are emitting a predetermined light intensity penetrating through the skin; and
(b) aligning the treatment channel with a vein which is highlighted by the light emitters for visualizing the vein under the skin.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of a vein position locating device according to a preferred embodiment of the present invention.

FIG. 2 is a perspective view of the vein probing head according to the above preferred embodiment of the present invention.

FIG. 3 is a schematic diagram of the vein locating device according to the above preferred embodiment of the present invention, illustrating the interaction between the vein probing head, the light transmission cable, and the operational control unit.

FIG. 4 illustrates the vein highlighted by the light emitters of the vein position locating device according to the above preferred embodiment of the present invention.

FIG. 5 is an alternative mode of the vein probing head according to the above preferred embodiment of the present invention.

FIG. 6 is a schematic flow diagram of a method of locating a vein under a user's skin according to the above preferred embodiment of the present invention.

### DETAILED DESCRIPTION OF THE PREFERRED EMBODIMENT

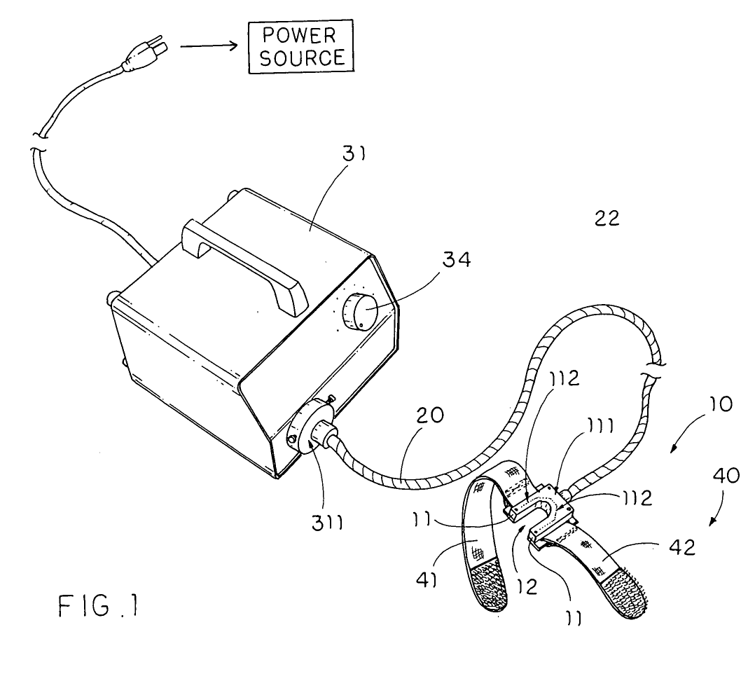

Referring to FIG. 1 of the drawings, a vein position locating device according to a preferred embodiment of the present invention is illustrated, in which the vein position locating device comprises a vein probing head 10, a light transmission cable 20, and an operational control unit 30.

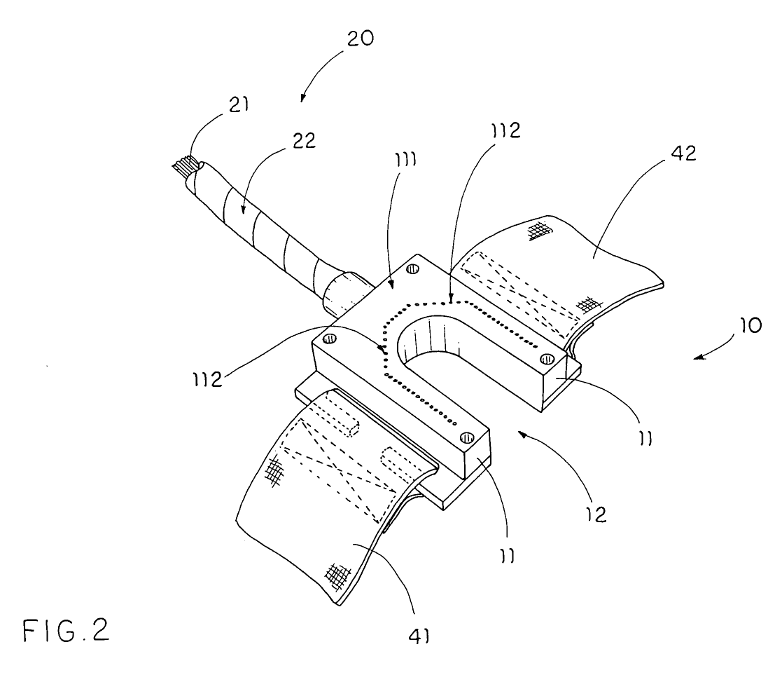

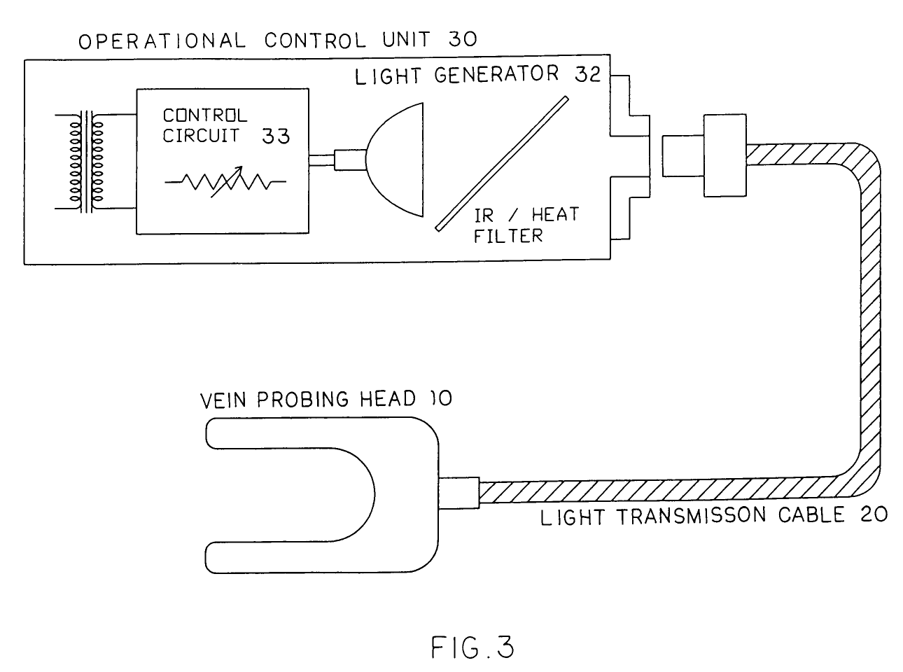

Referring to FIGS. 2 to 4 of the drawings, the vein probing head 10 comprises two light emitters 11 spacedly and opposedly positioned apart from each other to define a treatment channel 12 therebetween, wherein the vein probing head 10 is adapted for contacting on a skin surface 1 of a user so that each of the light emitters 11 is adapted for emitting a light beam of a predetermined intensity to penetrate through the user's skin surface 1 so as to visualize a vein 2 thereunder, wherein the vein probing head 10 is arranged for moving on the skin surface 1 of the user until the vein 2 is aligned in the treatment channel 12 between the two light emitters 11. As a result, the vein 2 is located for further medical treatment, such as an injection.

According to the preferred embodiment, each of the light emitters 11 is elongated in shape wherein a rear portion of each of the light emitters 11 is transversely and integrally extended towards each other to form a U-shaped structure which defines the treatment channel 12 between the two light emitters 11. Furthermore, each of the light emitters 11 has a predetermined thickness with respect to the skin's surface 1 so that a maximum amount of light can be trapped in the treatment channel 12.

Moreover, each of the light emitters 11 has a flat bottom emitting surface 111 allowing the light beam to pass through the emitting surface 111 of the vein probing head 10 for penetrating through the skin surface 1 of the user so as to highlight the vein 2 thereof. Accordingly, the emitting surfaces 111 of the light emitters 11 are adapted to smoothly and slidably move along a user's skin surface 1 for accurately and rapidly locating the vein 2 as well.

As shown in FIG. 2, each of the light emitters 11 has a plurality of light emitting holes 112 spacedly formed on the emitting surface 111 thereof such that when the vein probing head 10 is contacted on the skin surface of the user, the light beams are adapted to pass through the light emitting holes 112 for penetrating through the skin surface 1 of the user to highlight the vein 2 thereunder.

The operational control unit 30 comprises a portable casing 31, having a light outlet 311, a light generator 32 disposed therein for generating the light beam which is arranged to be transmitted to the vein probing head 10 through the light transmission cable 20, and a control circuit 33 electrically connected to the light generator 32 for controlling a light intensity delivered thereby. Moreover, a control switch 34 is provided on the portable casing 31 and is electrically connected with the control circuit 33 for allowing the user of the present invention to selectively adjust a light intensity generated by the light generator 32.

In other words, the light generator 32 and the control circuit 33 are received in the portable casing 31 while the light transmission cable 20 is detachably engaged with the light outlet 311 to communicatively connect with the light generator 32. Therefore, the vein position device is embodied as a portable device that the user is able to carry the vein position device anywhere. However, the vein position device can be built-in with other medical device such as blood pressure meter.

The light generator 32 is preferably embodied as a regular halogen lamp which is capable of generating light of predetermined level of light intensity. Alternatively, the light generator 32 may be embodied as a Light Emitting Diode (LED) the intensity of which is electronically controlled by the control circuit 33.

The light transmission cable 20 is extended from the vein probing head 10 to connect with the operational control unit 30 which is electrically connected with a power source, and is adapted to generate light beams which are to be transmitted to the vein probing head 10 through the light transmission cable 20.

Referring to FIG. 2 of the drawings, the light transmission cable 20 comprises a plurality of light transmission fibers 21, such as a plurality of regular optical fibers, each of which has a predetermined light transmitivity for transmitting light beams generated from the operational control unit 30 to the vein probing head 10. Moreover, the light transmission fibers 21 are flexible so that the vein probing head 10 is capable of freely moving on the user's skin surface 1 for locating the vein 2.

Accordingly, the light transmission cable 20 further comprises an outer protective coat 22 embedding the light transmission fibers 21 wherein the outer protective coat 22 is made inadmissible to light so that there should be no significant light intensity loss along the light transmission cable 20. In other words, the outer protective coat 22 is adapted to prevent the leakage of the light energy during the transmission from the light generator 32 to the vein probing head 10.

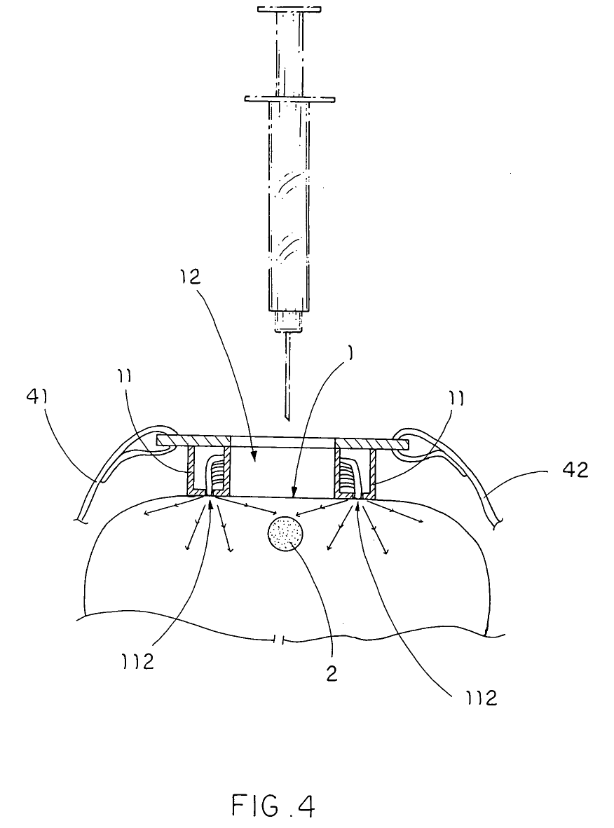

Generally speaking, the vein 2 is located approximately 0.5 inch under the skin. Since a plurality of pores are formed on the skin surface 1 of the user, the light beam from the vein probing head 10 is able to pass through the pores to penetrate through the skin surface 1 of the user so as to reach the vein 2 thereunder, as shown in FIG. 4. Therefore, the vein 2 will be easily highlighted once the vein probing head 10 is contacted with the skin surface I of the user. Accordingly, the vein 2 will be shadowed when the light beams penetrate the skin surface 1 of the user. In other words, the darken portion on the skin surface 1 of the user is illustrated the location of the vein 2 during the operation of the vein probing head 10.

As a result, it is worth pointing out that by adjusting a light intensity of the light generator 32, the light beams emitted by the light emitters 11 of the vein probing head 10 can be selectively adjusted for allowing the light beam to penetrate through the skin surface 1 to highlight the vein 2. In other words, the light emitted by the light emitters 11 can be adjusted to best visualize the user's vein 2.

The operation of the present invention is as follows: when the user switches on the operational control unit 30, the light generator 32 is activated to generate light beams of a predetermined light intensity. The light then passes through the light transmission cable 20 and reaches the vein probing head 10. Afterwards, the user has to place the vein probing head 10 on his/her body skin surface 1 and move and align the vein probing head 10 on the skin surface 1 until the vein 2 is highlighted and visualized in the treatment channel 12. After that, the user may perform his/her desired medical treatment, such as an injection, to the vein 2 in question.

Where the user finds difficulty in locating the vein 2, he/she might adjust the light intensity by switching the control switch 34 of the operational control unit 30 in order to acquire a satisfactory light intensity. In other words, when the light is not bright enough to penetrate the skin so as to visualize the vein 2, the user may increase the light intensity by adjusting the control switch 34 such that a satisfactory visualization of the vein 2 can be achieved.

It is worth mentioning that because the vein 2 is aligned in the treatment channel 12 which is illuminated by the two light emitters 11 wherein the light emitted therefrom is significantly trapped inside the treatment channel 12 for accumulating a sufficient amount of light to highlight the vein 2.

Moreover, the vein locating device 1 further comprises a means 40 for retaining the vein 2 to align within the treatment channel 12. The retaining means 40 comprises first and second fasteners 41, 42 extended from two outer sides of the vein probing head 10 for detachably fastening the vein probing head 10 with a body of the user, such as the user's arm.

Accordingly, the first and second fasteners 41, 42 are embodied as two elastic fabric attached to two outer sides of the light emitters 11 respectively wherein each of the first and second fasteners 41, 42 has a fastening surface in such a manner that the fastening surface of the first fastener 41 is adapted to overlappedly and detachably fasten on the fastening surface of the second fastener 42 so as to detachably fasten the vein probing head 10 on the user's body, such as the user's arm. Therefore, once the vein 2 is highlighted by the vein probing head 10, the first and second fasteners 41, 42 are fastened with each other to retain the vein probing head 10 in position that the vein 2 is aligned between the light emitters 11. Hook and loop fasteners are preferably provided on the fastening surfaces of the first and second fasteners 41, 42 to provide a quick and easy attachment of the retaining means 40.

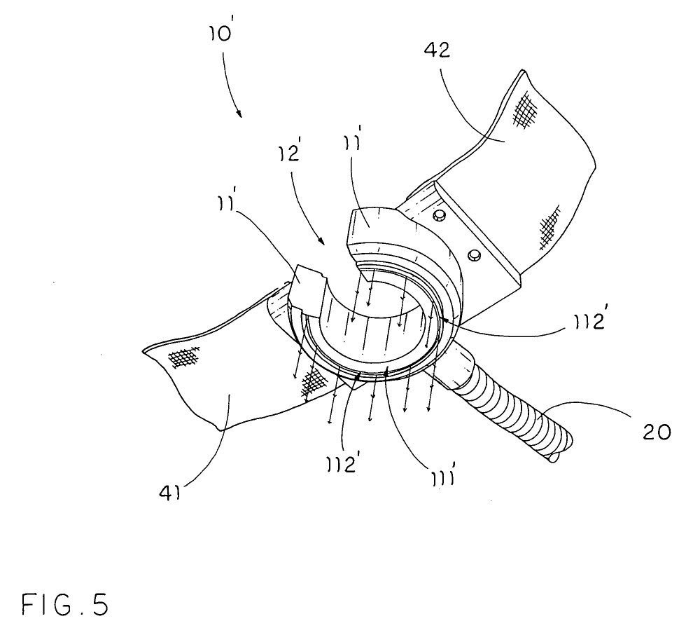

FIG. 5 illustrates an alternative mode of the vein locating device of the present invention. Each of the light emitters 11′ is semi-circular in shape and is integrally extended towards each other to form a circular cross section in which the treatment channel 12′ is defined within the two light emitters 11′, i.e. the circular vein probing head 10′.

Each of the light emitters 11′ has a flat bottom emitting surface 111′ allowing the light beam to pass through the emitting surface 111′ of the vein probing head 10′ for penetrating through the skin surface 1 of the user so as to highlight the vein 2 thereof. Accordingly, the emitting surfaces 111′ of the light emitters 11′ are adapted to smoothly and slidably move along a user's skin surface 1 for accurately and rapidly locating the vein 2 as well.

As shown in FIG. 5, each of the light emitters 111′ has a transparent light guiding portion 112′ provided on the emitting surface 111′ thereof such that when the vein probing head 10′ is contacted on the skin surface 1 of the user, the light beams are adapted to pass through the light guiding portion 112 for penetrating through the skin surface 1 of the user to highlight the vein 2 thereunder.

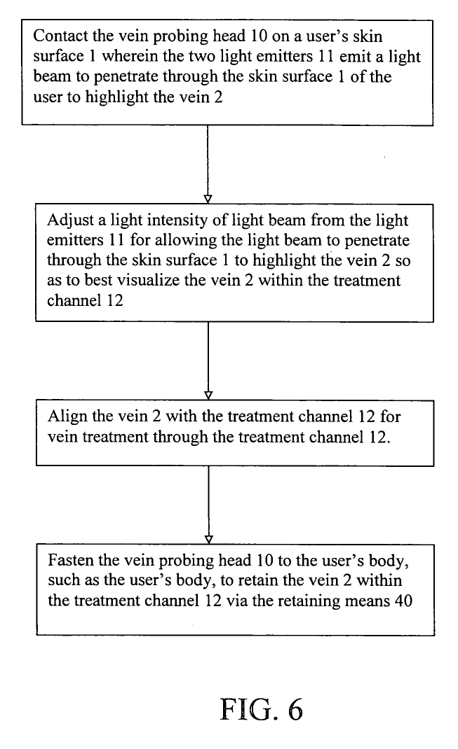

Referring to FIG. 6 of the drawings, a method of locating a vein 2 under a user's skin using the vein locating device 1 according to the preferred embodiment of present invention is illustrated. The method comprises the following steps.

(1) Contact the vein probing head 10 on a user's skin surface 1 wherein the two light emitters 11 emit a light beam to penetrate through the skin surface 1 of the user to highlight the vein 2.

(2) Align the vein 2 with the treatment channel 12 for vein treatment through the treatment channel 12.

As the light emitted by the light emitters 11 is adjustable, in step (1), the vein locating method further comprises a step of adjusting a light intensity of light beam from the light emitters 11 for allowing the light beam to penetrate through the skin surface 1 to highlight the vein 2 so as to best visualize the vein 2 within the treatment channel 12.

In step (2) above, the aligning process is preferably accomplished by slidably moving the vein probing head 10 on the body's skin surface 1 so as to locate the vein 2. In case that the light intensity is not sufficient, the user may increases the light intensity through the control switch 34 of the operational control unit 30 so as to make the visualization of the vein 2 to be clearer.

Once the vein 2 has been successfully located, fastening of the vein probing head 10 to the body, such as the user's arm, may be needed for more convenient medical treatment to the visualized vein 2. Accordingly, in step (2), the vein locating method may further comprise a step of fastening the vein probing head 10 to the user's body, such as the user's body, to retain the vein 2 within the treatment channel 12 via the retaining means 40.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

1. A vein position locating device, comprising:
a vein probing head which comprises two light emitters spacedly and opposedly apart from each other to define a treatment channel therebetween, wherein said vein probing head is adapted for contacting on a skin surface of a user so that each of said light emitters is adapted for emitting a light beam to penetrate through said skin surface of said user so as to visualize a vein thereunder, wherein said vein probing head is arranged for slidably moving on said skin surface of said user until said vein is aligned in said treatment channel between said two light emitters for vein treatment.

2. The vein position locating device, as recited in claim 1, wherein each of said light emitters has a plurality of light emitting holes spacedly formed on a bottom emitting surface of said light emitter such that said light beams pass through said light emitting holes for penetrating through said skin surface of said user to highlight said vein thereunder when said bottom emitting surfaces of said light emitters contact with said skin surface of said user.

3. The vein position locating device, as recited in claim 1, wherein each of said light emitters has a transparent light guiding portion formed on a bottom emitting surface of said light emitter such that said light beams pass through said light guiding portion for penetrating through said skin surface of said user to highlight said vein thereunder when said bottom emitting surfaces of said light emitters contact with said skin surface of said user.

4. The vein position locating device, as recited in claim 1, further comprising an operational control unit comprising a light generator for generating said light beam and a light transmission cable connecting said light generator with said vein probing head for transmitting said light beam from said light generator to said light emitters.

5. The vein position locating device, as recited in claim 2, further comprising an operational control unit comprising a light generator for generating said light beam and a light transmission cable connecting said light generator with said vein probing head for transmitting said light beam from said light generator to said light emitters.

6. The vein position locating device, as recited in claim 3, further comprising an operational control unit comprising a light generator for generating said light beam and a light transmission cable connecting said light generator with said vein probing head for transmitting said light beam from said light generator to said light emitters.

7. The vein position locating device, as recited in claim 4, wherein said operational control unit further comprises a control circuit electrically connected to said light generator to selectively adjust a light intensity of said light beam generated by said light generator towards said vein probing head for allowing said light beam to penetrate through said skin surface to highlight said vein.

8. The vein position locating device, as recited in claim 5, wherein said operational control unit further comprises a control circuit electrically connected to said light generator to selectively adjust a light intensity of said light beam generated by said light generator towards said vein probing head for allowing said light beam to penetrate through said skin surface to highlight said vein.

9. The vein position locating device, as recited in claim 6, wherein said operational control unit further comprises a control circuit electrically connected to said light generator to selectively adjust a light intensity of said light beam generated by said light generator towards said vein probing head for allowing said light beam to penetrate through said skin surface to highlight said vein.

10. The vein position locating device, as recited in claim 7, wherein said operational control unit further comprises a portable casing having a light outlet, wherein said light generator and said control circuit are received in said portable casing while said light transmission cable is detachably engaged with said light outlet to communicatively connect with said light generator.

11. The vein position locating device, as recited in claim 8, wherein said operational control unit further comprises a portable casing having a light outlet, wherein said light generator and said control circuit are received in said portable casing while said light transmission cable is detachably engaged with said light outlet to communicatively connect with said light generator.

12. The vein position locating device, as recited in claim 9, wherein said operational control unit further comprises a portable casing having a light outlet, wherein said light generator and said control circuit are received in said portable casing while said light transmission cable is detachably engaged with said light outlet to communicatively connect with said light generator.

13. The vein position locating device, as recited in claim 11, wherein said light transmission cable comprises at least a light transmission fiber having a predetermined light-transmitivity for transmitting said light beam generated by said light generator to said vein probing head.

14. The vein position locating device, as recited in claim 12, wherein said light transmission cable comprises at least a light transmission fiber having a predetermined light transmitivity for transmitting said light beam generated by said light generator to said vein probing head.

15. The vein position locating device, as recited in claim 1, further comprising means for retaining said vein to align within said treatment channel.

16. The vein position locating device, as recited in claim 13, further comprising means for retaining said vein to align within said treatment channel.

17. The vein position locating device, as recited in claim 14, further comprising means for retaining said vein to align within said treatment channel.

18. A method of locating a vein under a skin surface of a user by a vein position locating device which comprises a vein probing head comprising two spaced apart light emitters to define a treatment channel therebetween, wherein said vein position locating method comprises the steps of:
(a) contacting said vein probing head on said skin surface of said user wherein said two light emitters emit a light beam to penetrate through said skin surface of said user to highlight said vein; and
(b) aligning said vein with said treatment channel for vein treatment through said treatment channel.

19. The method as recited in claim 18, in step (a), further comprising a step of adjusting a light intensity of said light beam from said light emitters for allowing said light beam to penetrate through said skin surface to highlight said vein so as to best visualize said vein within said treatment channel.

20. The method as recited in claim 19, in step (b), further comprising a step of fastening said vein probing head to said user's body to retain said vein within said treatment channel.
